(19) United States
(12) Patent Application Publication
(10) Pub. No.: US 2011/0184502 A1
(43) Pub. Date: Jul. 28, 2011

(54) HEATING ELEMENT WITH THERMAL FUSE

(75) Inventor: Mark H. BRUDER, Alpharetta, GA (US)

(73) Assignee: BRUDER HEALTHCARE COMPANY, Alpharetta, GA (US)

(21) Appl. No.: 13/015,125

(22) Filed: Jan. 27, 2011

Related U.S. Application Data
(60) Provisional application No. 61/298,795, filed on Jan. 27, 2010.

(51) Int. Cl. A61F 7/08; H05B 3/34; H05B 1/02

(52) U.S. Cl. 607/114; 219/528; 219/494

(57) ABSTRACT

A heating pad for providing heat that comprises a heating element having an outer covering, an electrical and control assembly, and an inner sheet placed within the outer covering of the heating element. The electrical and control assembly includes a power cord and a controller switch that is attached to the heating element. The inner sheet houses a heating filament thereon. The heating filament has at least one thermostat attached along the heating filament for monitoring the temperature of the filament and at least one thermal fuse attached along the heating filament for preventing the filament from overheating.

### CROSS-REFERENCE TO RELATED APPLICATION

This application is related to U.S. Provisional Patent Application Ser. No. 61/298,795, filed Jan. 27, 2010, which is hereby incorporated herein by reference in its entirety.

### TECHNICAL FIELD

The present invention relates generally to the field of heating devices, and more particularly to a personal electrical heating device having a sealed heating panel with one or more electrical shutoff elements.

### BACKGROUND

Heating devices are utilized in various applications for personal comfort, analgesic therapy, equipment protection and other purposes. Examples include electric heating pads, electric blankets, bed warmers, heated car seats, warming trays, plate warmers and the like. Such devices commonly include one or more electrically resistive heating elements and a thermostat for controlling the degree of heating.

In therapeutic applications, personal electric heating elements can be used to heat or warm a particular part of a user's body to provide analgesia, improve circulation, for patient comfort, to enhance flexibility or mobility, or for other treatment purposes. For example, different human conditions can cause pain in different areas on a user's body which can be relieved in some circumstances by thermal treatment, either alone or in combination with physical therapy or other treatment regimens. Heated thermal treatment is commonly carried out by applying a heating pad or other warm object to a subject's body surface to lessen discomfort or pain the user is experiencing.

Heating pads can be electrically heated, heated by chemical reaction, or externally heated such as by placing a pad or compress in a heated water bath. Instances of electric heating pads malfunctioning and causing discomfort and/or injury to users have been reported. As a result, safety concerns have led many users or potential users to disfavor or even prohibit use of some types of electric heating pads. Alternate forms of heating, however, have also been found to have their own disadvantages.

Additionally, some previously known heating pads have provided for moist heat therapy application by applying water to a pad cover. Adding water to electrical products, however, presents consumer safety concerns. Furthermore, some electric heating pads produce moisture using intense heat to release stored moisture from the pad cover. A portion of such electric heating pads utilize intense heat reaching temperatures of up to 190°-212° F., which may cause patient discomfort or even burning. As such, previously known therapeutic devices for application of moist heat have not been found fully satisfactory by many users.

Thus it can be seen that needs exist for improved heating devices and methods. It is to the provision of improved heating methods and heating pad devices that the present invention is primarily directed.

### SUMMARY

The present invention provides improved heating devices and methods. The heating devices and methods of the invention may be utilized in connection with a variety of products for different intended applications, including without limitation, electric heating pads, electric blankets, bed warmers, heated car seats, warming trays, plate warmers and the like. U.S. Pat. No. 5,432,322 to Ingram et al is hereby incorporated herein by reference, and shows an example electric heating pad assembly. In example forms, the heating device of the present invention incorporates one or more thermal fuses that provide an electrical disconnect in the event of overheating beyond a specified temperature, thereby improving safety.

In an example embodiment, the invention relates to a heating pad including a heating element having an outer covering, an electrical and control assembly, and an inner heating element sealed within an encasement for placement within the outer covering. The electrical and control assembly includes a power cord and a controller switch that is attached to the heating element. The inner heating element includes at least one resistive heating member such as a printed trace or filament, at least one thermostat or thermistor for monitoring and controlling the temperature of the resistive heating member, and at least one thermal fuse for preventing the heating member from overheating. Because previously known heating pads have incorporated thermostats for temperature control, and because such products are commonly subject to commodity pricing pressures, those skilled in the art would heretofore have considered it counterintuitive to utilize thermistors in a heating pad. Similarly, those of skill in the art have not previously considered the use of thermal fuses applicable to heating pads.

In another aspect, the invention relates to a method of therapy including providing a heating pad comprising a heating element, an outer covering and a heating filament with at least one thermal fuse for deactivating the heating element in the event of a thermal overload; applying the heating pad to a subject; and actuating the thermal fuse in the event of a thermal overload to prevent injury to the subject.

In another aspect, the invention relates to a heating device including at least one resistive heating element, at least one flexible conductor for delivering electrical current to the resistive heating element, and a thermal disconnect for interrupting delivery of electrical current when a maximum threshold temperature is exceeded.

In still another aspect, the invention relates to a heating device for application of moist heat therapy without application of liquid water to the device. The device preferably includes a moisture absorbent material for absorbing ambient water vapor when unheated, and releasing contained water vapor upon heating. The device preferably further includes timing and control circuitry for delivering moist heat therapy at one or more prescribed temperature points and duration periods.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 shows a heating device according to an example embodiment of the present invention.

FIG. 2 is a top view of a portion of the heating device of FIG. 1, including an inner heating element and panel.

FIG. 3 is a bottom view of the inner heating element and panel shown in FIG. 2.

FIG. 4 is a top view of a portion of a heating device according to another example embodiment of the present invention.

FIG. 5 is an electrical schematic showing circuitry of a heating element according to another example embodiment of the present invention.

FIG. 6 is a flowchart showing sequences of operation of a heating device according to example embodiments of the invention.

FIG. 7 shows a control device for a heating device, including moist heat therapy, according to another example embodiment of the invention.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

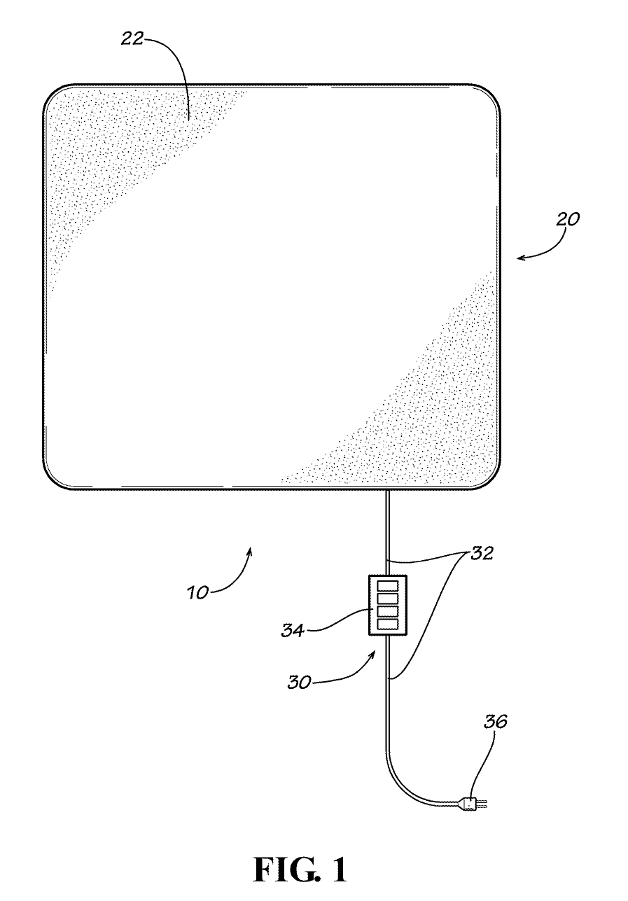

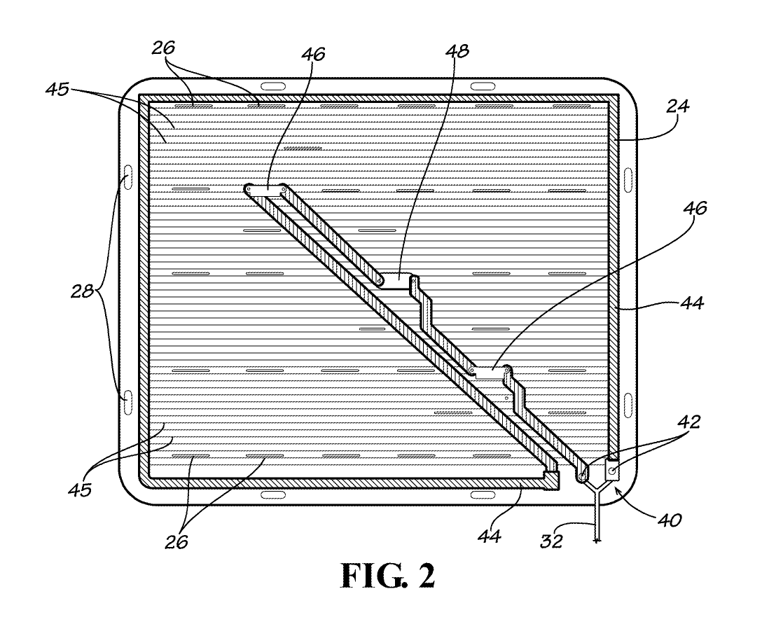

With reference now to the drawing figures, wherein like reference numbers represent corresponding parts throughout the several views, FIG. 1 shows a heating device 10 according to a first example embodiment according to the present invention. The heating device 10 generally includes a heating pad or body 20, an electrical and control assembly 30, and a power cord 36 for connecting the heating device to an electrical outlet. In example embodiments, the heating pad 20 includes an outer shell or covering 22 and an inner heating panel 24 (as seen in FIG. 2) for placement within the outer covering. Preferably, the outer covering 22 is a rectangular-shaped pouch operable to removably envelop the panel 24 and can be secured thereto with one or more conventional fasteners, such as a zipper, hook and loop fastener, snap, button, etc. Alternatively, the covering 22 can be permanently secured to the underlying panel 24 or can be eliminated as desired. The outer covering can be fabricated from various materials including, but not limited to, fleece, cotton, polyester, nylon, and/or various polymers. However, in preferred example embodiments, the outer covering 22 is formed from woven cotton fibers (and/or other natural fibers), which is a naturally hydrating material. As such, the outer covering 22 absorbs ambient water vapor from the surroundings and releases this moisture when heated to provide moist heat therapy. In alternative example embodiments, the covering 22 can be circular, triangular, elliptical or otherwise shaped as desired.

The electrical and control assembly 30 includes a power cord 32, a control switch 34, and an electrical plug 36 located at the distal end of the power cord. In depicted embodiments, the control switch 34 is located along the power cord 32, but in alternate embodiments, the control switch can be located on the heating pad 20, the covering 22, or at any other suitable location in connection with the heating device 10. Generally, the control switch 34 allows a user to operate the heating device 10 and regulate the heat output of the same. For example, the control switch 34 can allow a user to select a desired heat setting, such as by choosing between “Low,” “Medium,” or “High” heat settings or an adjustable numerical-based heat setting. The electrical and control assembly 30 can be manufactured to accept 110V, 220V, or any other type of electrical input as desired.

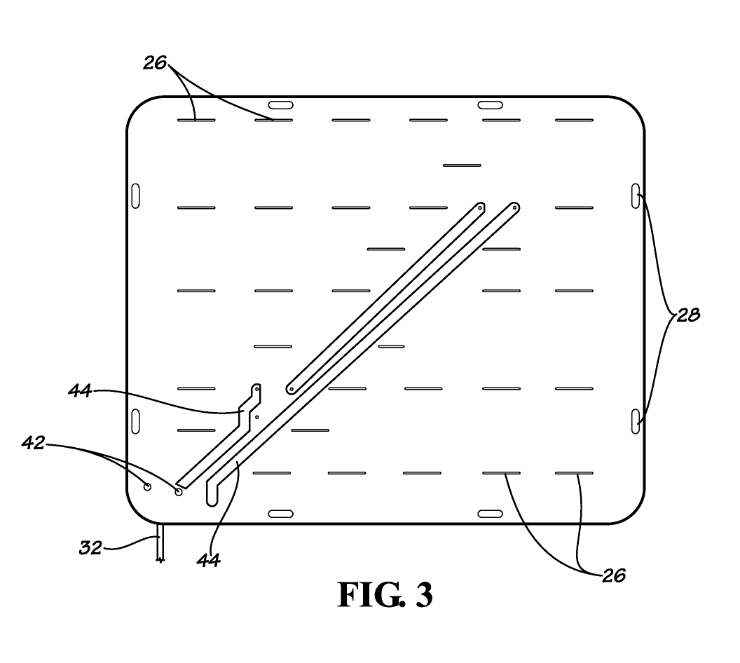

An example embodiment of the inner heating panel 24 is depicted in FIGS. 2-3. As shown therein, the panel 24 is generally rectangular in shape and has a length and height that are significantly larger than its width (or thickness). In alternative embodiments, the panel 24 can be shaped and sized as desired. The inner panel 24 can be formed of any durable, flexible, and heat-resistant material, including medical grade silicone, Mylar, polyethylene, or other plastics, polymers, etc. In preferred example embodiments, the panel 24 is formed from a Mylar base as described in more detail below. As seen in the drawing figures, the panel 24 can optionally include a plurality of stress-relieving slots/hinges 26 and/or a plurality of connecting apertures 28. When implemented, the slots 26 are generally positioned in one or more rows along the inner panel 24 to provide improved flexibility and compliance to the contour of subjects to which the heating device 10 is applied. The slots 26 are formed by the absence of at least an elongated portion of the panel 24, which allows the inner panel 24 to bend and flex more easily about the slots. The optional connecting apertures 28 can be located near the periphery of the inner panel 24 to allow attachment of the outer covering 22 to the panel and/or an encapsulation layer onto the same. For example, in additional example embodiments an outer encapsulation layer of medical grade silicone and/or Mylar can be applied over an inner Mylar base to form a hermetically sealed encapsulation of the internal circuitry and/or electrical components.

FIGS. 2 and 3 further depict an example configuration of a heating element assembly 40 of the inner panel 24. The heating element assembly 40 comprises a pair of connection terminals 42, at least one flexible conductor 44, a plurality of resistive heating elements 45, one or more thermal control components 46 such as thermostats or thermistors, and at least one thermal electrical disconnect 48 such as a thermal fuse, thermal switch or thermal reset. The flexible conductor(s) 44 and/or the resistive heating elements 45 are preferably printed in conductive or semiconductive inks or traces onto a substrate such as a Mylar base, but in alternate forms can comprise wires, channels of encased electric or thermal conducting liquid, and/or other conductive means. For example, the flexible conductor(s) 44 and conductors for communication between the thermal control components and the system's control circuitry can comprise a silver conductive paste such as SH-8301 by Eisho Electronic Materials Co., Ltd. of Shenzhen, China; and the resistive heating elements 45 can comprise a carbon ink such as Jelcon CH-10 by Jujo Chemical Co., Ltd. of Tokyo, Japan. In this depicted embodiment, the flexible conductor(s) 44 extend along the periphery of the inner panel 24 and diagonally into the inner panel to distribute current to the resistive heating elements 45.

As depicted in FIG. 2, at least one, and preferably a pair of thermostats are provided as thermal control components 46 at spaced apart positions along the surface of the heating element assembly 40, to provide a more even heat distribution. The thermostats are in electronic communication with control circuitry of the control assembly 30 to control the delivery of electricity to the heating elements and thereby regulate the application of heat. A thermal cutoff or thermal fuse is provided as a thermal electrical disconnect 48 to shut off the delivery of current to the heating elements if a maximum threshold temperature is exceeded, for example to prevent burning the subject or damaging equipment. Example thermal fuses adaptable for use in connection with the heating device of the present invention include the Vicfuse (Victors Industrial Co., Ltd., Hong Kong). In example embodiments, the thermal fuse or other thermal electrical disconnect 48 has a shutoff temperature of at least 100° F., between 95-250° F., between 110-195° F., between 120-155° F., between 104-113° F., about 125° F., or other specified temperature based on the intended application. The thermostats 46 and the thermal fuse 48 are preferably positioned along the diagonal portion of the flexible conductor 44, with the thermal fuse 48 positioned between the thermostats 46. In other example embodiments, the one or more thermostats 46 and thermal fuse 48 can be alternatively positioned on the heating element assembly as desired.

In the event that the heating element assembly 40 overheats or spikes in temperature, the thermal fuse 48 attached thereto melts or breaks open to disconnect the electrical circuit and disrupt the electrical current flowing through the filament. The pathway for the electricity to flow would therefore be interrupted, and the heating filament 44 would cease to rise in temperature. The controller circuit in the control switch 34 can be designed to detect when the electrical current is cut off by the thermal fuse 48 and then turn the heating pad 10 off.

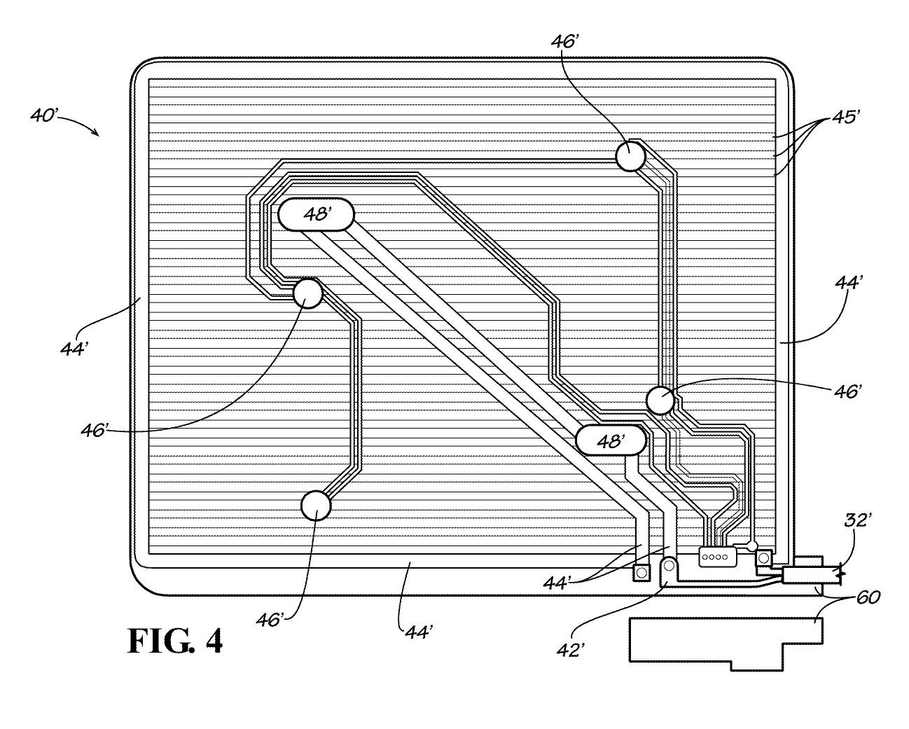

FIG. 4 shows another example embodiment of a heating element assembly 40′, including a pair of connection terminals 42′, at least one flexible conductor 44′, a plurality of resistive heating elements 45′, four thermistors 46′, and two thermal fuses 48′. A contact cover 60 (shown disassembled for clarity) protects the connection terminals 42′ and cord 32′ from damage during use. The thermistors 46′ are positioned in separate quadrants of the assembly, and communicate back to the control circuitry to maintain an even temperature across substantially the entire surface area of the heating device. Optionally, the control circuitry individually adjusts delivery of current to the resistive heating elements within each quadrant of the heating element assembly in response to that quadrant's thermistor. The thermal fuses are installed in series along the flexible conductor circuit 44′ as a redundant safety measure, whereby failure of either will shut off delivery of current to the resistive heating elements 45′ and stop further heating.

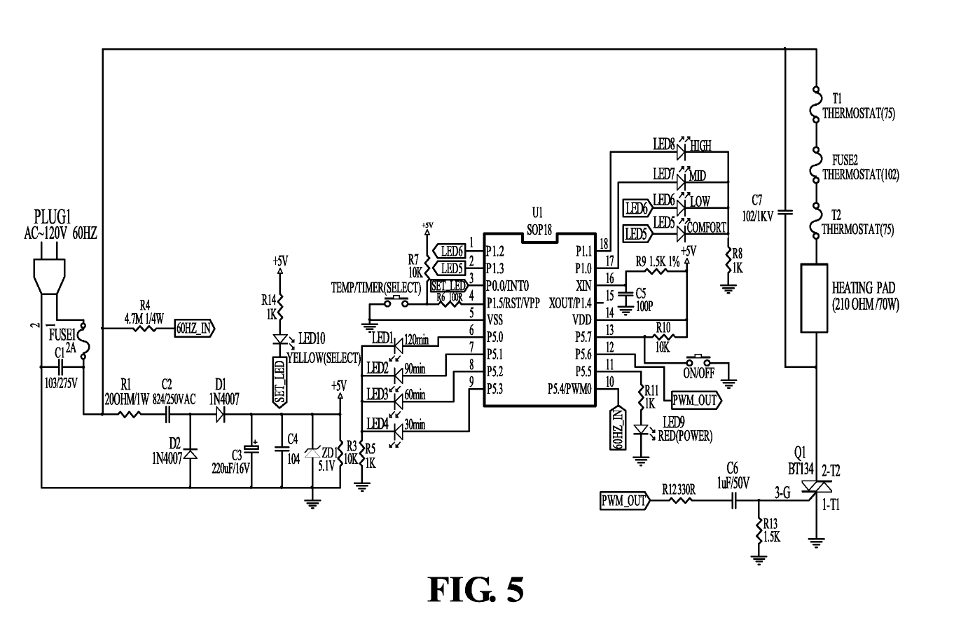

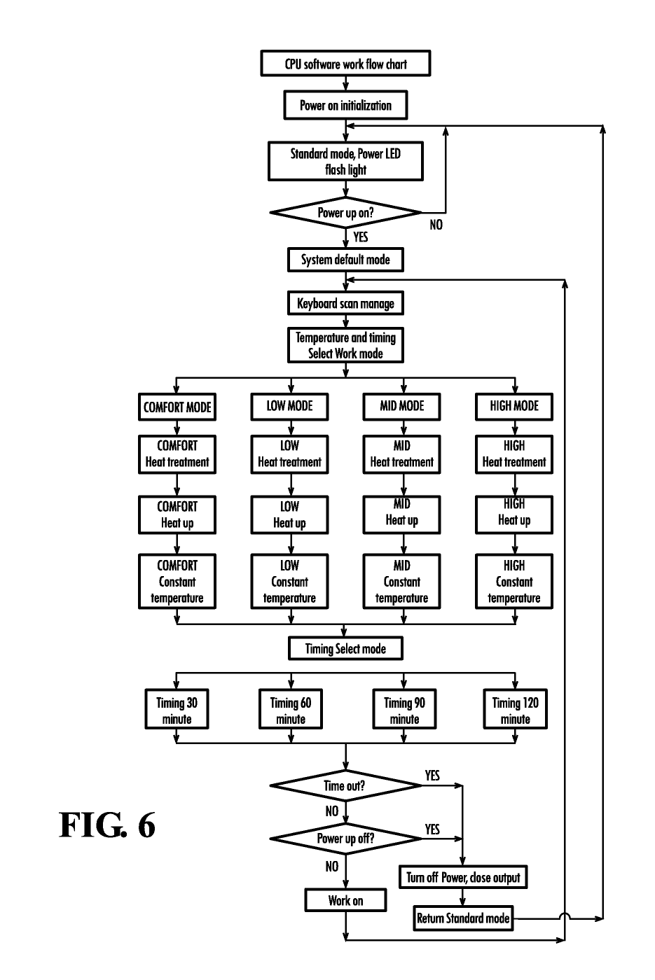

FIG. 5 shows a schematic diagram of the electronic circuitry of a heating device according to another example embodiment of the invention. The device includes a microprocessor, at least one input device such as switch(es) and/or control button(s) for receiving instructions from a user, and at least one output such as a display or indicator lights for indicating the status of the device. FIG. 6 is a flowchart showing example modes of operation of a heating device according to an example form of the invention. In example methods of operation, a user can select from different heat application modes which can vary in terms of the duration of heat application, the temperature(s) of application, the rate of change of applied temperature(s) at different stages, and the like. Optionally, the device provides for automatic shutoff after a specified duration of heat application.

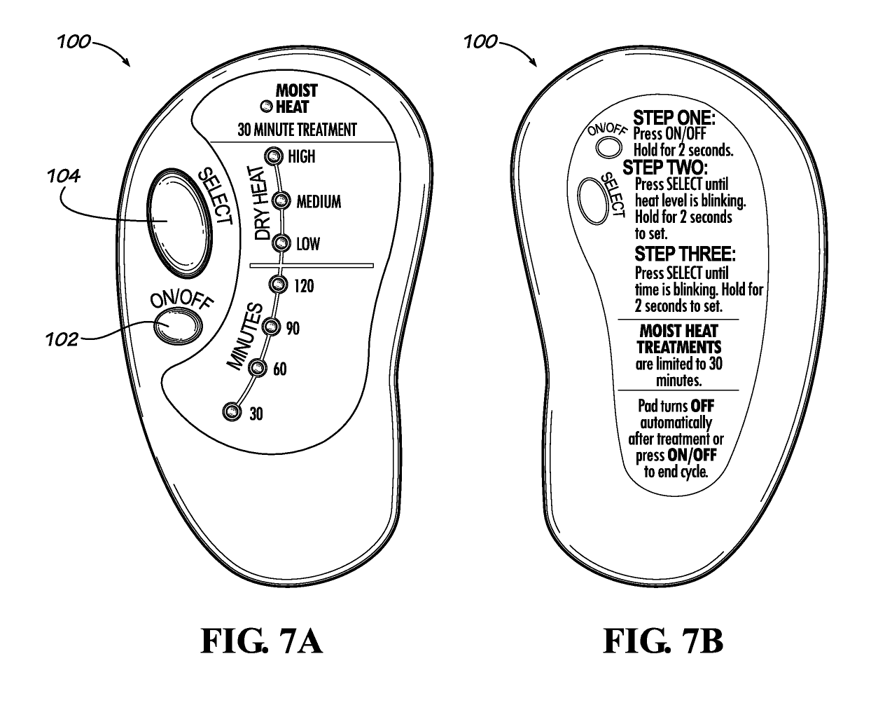

FIG. 7 shows a particular control device 100 for a therapeutic heating device including moist heat application according to another example embodiment of the present invention. The control device 100 includes push-button controls 102, 104 for powering the device on or off, and for selecting a therapeutic regimen from one or more pre-determined temperature and/or duration controlled regimens. For example, a use may select between 30 minute, 60 minute, 90 minute and 120 minute durations; dry heat application at low, medium or high temperature settings; and/or moist heat application. The control device 100 includes timing and control circuitry for controlling the duration and temperature at which heat is applied. In additional example embodiments, the control circuitry includes at least one CPU and/or microprocessor to allow the device to function in a plurality of preprogrammed operational modes as desired by a user. The at least one CPU/Microprocessor works by selectively and automatically regulating the flow of electricity to the device to follow a designed temperature/duration program.

For instance, in example modes of operation for moist heat application, the heating device 10 of the present invention is allowed to absorb water vapor from the ambient surroundings rather than require a user to add liquid water to a cover (as is typical for known heating pad devices). As previously discussed, example embodiments of the heating device 10 utilize an outer covering comprised of natural fibers, such as cotton fibers, that is exposed to ambient conditions for at least several minutes, and up to an hour or more before being heated. Actual exposure time for the covering depends on many factors including ambient air temperature and humidity. Once the covering 22 has absorbed the desired amount of ambient moisture, the device 10 is then energized and applied to a body surface of a subject for heat therapy. In commercial embodiments, the heating device 10 operating in “Moist Heat” mode, heats to about 155° F. within approximately seven minutes, maintains this temperature for several minutes, and then reduces the temperature to about 125° F. over a period of approximately twenty minutes before automatically shutting off. Upon the device 10 reaching about 155° F., moisture from the device is released to the treated body surface. In alternative embodiments, other time durations and/or temperature settings/ranges can be utilized in conjunction with moist heat applications. A significant advantage of the heating device 10 described herein is that a user desiring moist heat therapy is no longer required to add liquid water to an electrical heating device, which is both convenient for a user and also removes many of the safety concerns that accompany known devices. Additionally, the heating device 10 of the present invention is able to provide moist heat therapy without approaching intense temperatures that pose a risk of burning a user. In alternate embodiments, the device 10 can deliver moist heat therapy at temperature ranges of between about 110° F. to about 160° F., and more specifically between about 130° F. to about 160° F., and even more specifically between about 150° F. to about 160° F.

Example embodiments of the heating device 10 disclosed herein can also include a plurality of dry heat operational modes. For instance, in a first example operational mode, the heating device reaches a maximum temperature of between about 155° F. and about 160° F. within approximately seven minutes after user initiation. Once the maximum temperature has been reached, the heating device is maintained at this temperature for approximately eight minutes before it is gradually reduced to a temperature of between about 130° F. and about 140° F. Finally, in this first example operational mode, the heating device 10 automatically shuts off after a total operational time of about 30 minutes.

In a second example operational mode, the heating device 10 reaches a maximum temperature of between about 155° F. and about 160° F. within approximately seven minutes after user initiation. Once the maximum temperature has been reached, the heating device is maintained at this temperature for approximately eight minutes before it is gradually reduced to a temperature of between about 110° F. and about 120° F. over a period of approximately 15 minutes. Thereafter, the heating device 10 maintains this reduced temperature range for a period of 60 minutes before automatically shutting down after a total operational time of about 90 minutes.

In still a third example operational mode, the heating device 10 reaches a maximum temperature of between about 155° F. and about 160° F. within approximately seven minutes after user initiation. Once the maximum temperature has been reached, the heating device is maintained at this temperature for approximately eight minutes before it is gradually reduced to a temperature of between about 110° F. and about 120° F. over a period of approximately 15 minutes. Thereafter, the heating device 10 maintains this reduced temperature range for a period of 120 minutes before automatically shutting down after a total operational time of about 150 minutes.

In a fourth example operational mode, the heating device 10 reaches a maximum temperature range of between about 130° F. and about 140° F. within a seven-minute time period from user initiation and remains within this temperature range until the device automatically shuts off after a 30 minute working period.

In a fifth example operational mode, the heating device 10 reaches a maximum temperature of between about 130° F. and about 140° F. within approximately seven minutes after user initiation. Once the maximum temperature has been reached, the heating device is maintained at this temperature for approximately eight minutes before it is gradually reduced to a temperature of between about 110° F. and about 120° F. over a period of approximately 15 minutes. Thereafter, the heating device 10 maintains this reduced temperature range for a period of 60 minutes before automatically shutting down after a total operational time of about 90 minutes.

In a sixth example operational mode, the heating device 10 reaches a maximum temperature of between about 130° F. and about 140° F. within approximately seven minutes after user initiation. Once the maximum temperature has been reached, the heating device is maintained at this temperature for approximately eight minutes before it is gradually reduced to a temperature of between about 110° F. and about 120° F. over a period of approximately 15 minutes. Thereafter, the heating device 10 maintains this reduced temperature range for a period of 120 minutes before automatically shutting down after a total operational time of about 150 minutes.

In a seventh example operational mode, the heating device 10 reaches a maximum temperature of between about 110° F. and about 120° F. after user initiation and remains within this temperature range for approximately 120 minutes before automatically shutting down.

In an eight example operational mode, the heating device 10 reaches a maximum temperature of between about 100° F. and about 110° F. after user initiation and remains within this temperature range for approximately 120 minutes before automatically shutting down.

Commercial embodiments of the heating device 10 of the present invention utilize the foregoing example preprogrammed operational modes (and/or feature additional operational modes) to offer users safe and convenient therapeutic thermal programs to take the guesswork out of heat treatment. As such, a user is able to reliably utilize thermal therapy that complies with medically prescribed treatment parameters without being exposed to burn risks from the application of excessively high temperatures/durations.

While the invention has been described with reference to preferred and example embodiments, it will be understood by those skilled in the art that a variety of modifications, additions and deletions are within the scope of the invention, as defined by the following claims.

What is claimed is:

1. A heating pad comprising:
a heating element having an outer covering;
an electrical and control assembly for connection of the heating element to a power source; and
a heating filament within the heating element, the heating filament having at least one thermal fuse attached along the heating filament for preventing the filament from overheating.

2. The heating pad of claim 1, wherein the heating filament further comprises at least one thermostat.

3. The heating pad of claim 1, further comprising a thermistor.

4. The heating pad of claim 1, wherein the heating filament comprises conductive or semiconductive ink.

5. The heating pad of claim 2, further comprising a central processing unit in electrical communication with the heating filament.

6. The heating pad of claim 5, wherein the control assembly includes a plurality of preprogrammed operational modes.

7. The heating pad of claim 6, wherein a first preprogrammed operational mode provides for moist heat.

8. A method of therapy comprising:
providing a heating pad comprising a heating element, an outer covering and a heating filament with at least one thermal fuse for deactivating the heating element in the event of a thermal overload;
applying the heating pad to a subject; and
actuating the thermal fuse in the event of a thermal overload to prevent injury to the subject.

9. The method of therapy of claim 8, wherein the heating element comprises conductive ink.

10. The method of therapy of claim 9, wherein the outer covering releases moisture at a temperature of less than about 160° F.

11. A heating device comprising:
at least one resistive heating element;
at least one flexible conductor for delivering electrical current to the resistive heating element; and
a thermal disconnect for interrupting delivery of electrical current when a maximum threshold temperature is exceeded.

12. The heating device of claim 11, wherein the thermal disconnect is a thermal fuse.

13. The heating device of claim 11, further comprising a thermal control component.

14. The heating device of claim 13, wherein the thermal control component is a thermistor.

15. The heating device of claim 11, wherein the resistive heating element and the flexible conductor are printed on a base.

16. The heating device of claim 14, wherein the resistive heating element, the flexible conductor and the base are hermetically sealed by an outer cover.

17. The heating device of claim 16, wherein the base comprises Mylar and the outer cover comprises silicone.

18. The heating device of claim 15, wherein the base comprises a series of slots for improving flexibility.

19. The heating device of claim 11, further comprising an outer covering.

20. The heating device of claim 19, wherein the outer covering is removable.

21. The heating device of claim 19, wherein the outer covering comprises a naturally hydrating material that absorbs ambient water vapor and releases moisture during use to provide moist heat therapy.

22. The heating device of claim 21, wherein the outer covering releases moisture when exposed to temperatures of between about 150° F. and about 160° F.

23. The heating device of claim 11, further comprising a central processing unit in electrical communication with the at least one flexible conductor.

24. The heating device of claim 23, wherein the central processing unit is operable to execute at least one preprogrammed operational mode.

25. The heating device of claim 24, wherein a first preprogrammed operational mode is a moist heat therapeutic program lasting about 30 minutes.
